(19) United States
(12) Patent Application Publication
(10) Pub. No.: US 2026/0133156 A1
(43) Pub. Date: May 14, 2026

(54) MULTI-BIAS SCHEME FOR AN ELECTROCHEMICAL GAS SENSOR

(71) Applicant: QUALCOMM Incorporated, San Diego, CA (US)

(72) Inventors: Changting XU, San Jose, CA (US); Jiezhi YANG, San Jose, CA (US); Efthymios Philip PAPAGEORGIOU, Berkeley, CA (US); Jim Chih-Min CHENG, Fremont, CA (US); Kostadin Dimitrov DJORDJEV, Los Gatos, CA (US)

(21) Appl. No.: 18/948,060

(22) Filed: Nov. 14, 2024

(51) Int. Cl. G01N 27/413; G01N 33/00

(52) U.S. Cl. CPC G01N 27/413; G01N 33/0068

(57) ABSTRACT

Various aspects of the present disclosure generally relate to gas detection. In some aspects, a gas detection system may receive, via one or more gas apertures in an electrochemical device, a composition of one or more gases. The gas detection system may apply a set of bias voltage sub-cycles, wherein, for each bias voltage sub-cycle, an applied voltage alternates between a corresponding steady voltage and a plurality of time-varying voltages centered about the corresponding steady voltage. The gas detection system may detect at least one of a current or a voltage while applying the set of bias voltage sub-cycles. The gas detection system may identify at least one of a presence of each gas or a concentration of each gas based at least in part on the at least one of the detected current or the detected voltage. Numerous other aspects are described.

### FIELD OF THE DISCLOSURE

Aspects of the present disclosure generally relate to sensing and identification of gases by an electrochemical cell in conjunction with a sensing circuit and specifically relate to techniques, apparatuses, and methods associated with a multi-bias scheme for an electrochemical gas sensor.

### BACKGROUND

Given the dramatic changes in the earth's atmosphere, precipitated by industrialization and natural sources, as well as the dramatically increasing number of household and urban pollution sources, accurate and continuous air quality monitoring has become necessary to both identify the sources and warn consumers of impending danger. Tantamount to making real-time monitoring and exposure assessment a reality is the ability to deliver low cost, small form factor, and low power devices which can be integrated into the broadest range of platforms and applications. There are multiple methods of sensing distinct low-density materials such as gases. Common methods include gas chromatography, nondispersive infrared spectroscopy (NDIR), the use of metal oxide sensors, the use of chemiresistors, and/or the use of electrochemical sensors, among other examples.

In an electrochemical sensor, a sensor electrode (also known as a working electrode) contacts a suitable electrolyte. Gas spreads along the surface of and permeates through the electrode (e.g., if the electrode is porous) and contacts the electrolyte. The sensor electrode typically comprises a catalytic metal that reacts with the target gas and the electrolyte to release or accept electrons, which creates a signal current when the electrode is properly biased and/or when used in conjunction with an appropriate counter-electrode. The current is generally proportional to the amount of target gas contacting the sensor electrode.

By using a sensor electrode material and/or bias voltage that is appropriate for the particular gas to be detected, the concentration of the target gas in the ambient atmosphere can be determined from the sensing current. The sensitivity of an electrochemical cell to a particular gas may be impacted by the application of a bias voltage to that cell. Therefore, by applying a set of different biases to an electrochemical cell and comparing a recorded signal to a library of signals (e.g., in a look-up table), it is possible to ascertain the presence of, differentiate between, and/or quantify the occurrence of multiple gases in the environment of the sensor. By continuously and quickly ramping the bias applied to the cell, a single electrochemical cell may rapidly differentiate between multiple gases in its environment.

### SUMMARY

In some aspects, a method of gas detection performed by an electrochemical device includes receiving, via one or more gas apertures in the electrochemical device, a composition of one or more gases; applying, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage; detecting at least one of a current or a voltage while applying the set of bias voltage sub-cycles, resulting in at least one of a detected current or a detected voltage; and identifying at least one of a presence of each gas, of the one or more gases, or a concentration of each gas, of the one or more gases, based at least in part on the at least one of the detected current or the detected voltage.

In some aspects, a gas detection system includes an electrochemical device; one or more components, coupled to the electrochemical device, configured to cause the gas detection system to: receive, via one or more gas apertures in the electrochemical device, a composition of one or more gases; apply, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage; detect at least one of a current or a voltage while applying the set of bias voltage sub-cycles, resulting in at least one of a detected current or a detected voltage; and identify at least one of a presence of each gas, of the one or more gases, or a concentration of each gas, of the one or more gases, based at least in part on the at least one of the detected current or the detected voltage.

In some aspects, a non-transitory computer-readable medium storing a set of instructions for gas detection includes one or more instructions that, when executed by one or more processors of a gas detection system, cause the gas detection system to: receive, via one or more gas apertures in an electrochemical device, a composition of one or more gases; apply, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage; detect at least one of a current or a voltage while applying the set of bias voltage sub-cycles, resulting in at least one of a detected current or a detected voltage; and identify at least one of a presence of each gas, of the one or more gases, or a concentration of each gas, of the one or more gases, based at least in part on the at least one of the detected current or the detected voltage.

In some aspects, an apparatus for gas detection includes means for receiving, via one or more gas apertures in an electrochemical device, a composition of one or more gases; means for applying, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage; means for detecting at least one of a current or a voltage while applying the set of bias voltage sub-cycles, resulting in at least one of a detected current or a detected voltage; and means for identifying at least one of a presence of each gas, of the one or more gases, or a concentration of each gas, of the one or more gases, based at least in part on the at least one of the detected current or the detected voltage.

Aspects of the present disclosure may generally be implemented by or as a method, apparatus, system, computer program product, non-transitory computer-readable medium, and/or processing system as substantially described with reference to, and as illustrated by, this specification and accompanying drawings.

The foregoing paragraphs of this section have broadly summarized some aspects of the present disclosure. These and additional aspects and associated advantages will be described hereinafter. The disclosed aspects may be used as a basis for modifying or designing other aspects for carrying out the same or similar purposes of the present disclosure. Such equivalent aspects do not depart from the scope of the appended claims. Characteristics of the aspects disclosed herein, both their organization and method of operation, together with associated advantages, will be better understood from the following description when considered in connection with the accompanying drawings.

### BRIEF DESCRIPTION OF THE DRAWINGS

The appended drawings illustrate some aspects of the present disclosure but are not limiting of the scope of the present disclosure because the description may enable other aspects. Each of the drawings is provided for purposes of illustration and description, and not as a definition of the limits of the claims. The same or similar reference numbers in different drawings may identify the same or similar elements.

FIG. 1 is a diagram illustrating an example of an electrochemical device, in accordance with the present disclosure.

FIG. 2 is a diagram illustrating an example gas detection system that implements an electrochemical device, in accordance with the present disclosure.

FIG. 3 is a diagram illustrating an example associated with single bias driving scheme for an electrochemical gas sensor, in accordance with the present disclosure.

FIG. 4 is a diagram illustrating an example associated with a multi-bias driving scheme for an electrochemical gas sensor, in accordance with the present disclosure.

FIG. 5 is a diagram illustrating an example associated with calculating concentrations of gases using a multi-bias driving scheme for an electrochemical gas sensor, in accordance with the present disclosure.

FIG. 6 is a diagram illustrating examples associated with detecting presence of gases using a multi-bias driving scheme for an electrochemical gas sensor, in accordance with the present disclosure.

FIG. 7 is a diagram illustrating an example process performed, for example, at an electrochemical device or an apparatus of an electrochemical device, and/or a gas detection system or an apparatus of a gas detection system, in accordance with the present disclosure.

### DETAILED DESCRIPTION

Various aspects of the present disclosure are described hereinafter with reference to the accompanying drawings. However, aspects of the present disclosure may be embodied in many different forms. The present disclosure is not to be construed as limited to any specific aspect illustrated by or described with reference to an accompanying drawing or otherwise presented in this disclosure. Rather, these aspects are provided so that this disclosure will be thorough and complete, and will fully convey the scope of the disclosure to those skilled in the art. One skilled in the art may appreciate that the scope of the disclosure is intended to cover any aspect of the disclosure disclosed herein, whether implemented independently of or in combination with any other aspect of the disclosure. For example, an apparatus may be implemented or a method may be practiced using various combinations or quantities of the aspects set forth herein. In addition, the scope of the disclosure is intended to cover an apparatus having, or a method that is practiced using, other structures and/or functionalities in addition to or other than the structures and/or functionalities with which various aspects of the disclosure set forth herein may be practiced. Any aspect of the disclosure disclosed herein may be embodied by one or more elements of a claim.

Several aspects of gas detection systems will now be presented with reference to various methods, operations, apparatuses, and techniques. These methods, operations, apparatuses, and techniques will be described in the following detailed description and illustrated in the accompanying drawings by various blocks, modules, components, circuits, steps, processes, or algorithms (collectively referred to as “elements”). These elements may be implemented using hardware, software, or a combination of hardware and software. Whether such elements are implemented as hardware or software depends upon the particular application and design constraints imposed on the overall system.

Electrochemical devices, such as electrochemical sensors, may be capable of detecting a presence of a target gas and/or a concentration of a target gas in an environment. In an electrochemical sensor, a sensor electrode (also known as a working electrode) contacts a suitable electrolyte. Gas enters the sensor and contacts the electrolyte. The sensor electrode typically comprises a catalytic metal that reacts with the target gas and electrolyte to release or accept electrons, which creates a characteristic current in the electrolyte when the electrode is properly biased and when used in conjunction with an appropriate counter-electrode. The current is generally proportional to the amount of target gas contacting the sensor electrode.

By using a sensor electrode material and bias voltage that is appropriate for the particular gas to be detected, the concentration of the target gas in the ambient atmosphere can be determined from the sensing current. The sensitivity of an electrochemical cell to a particular gas may be impacted by the application of a bias voltage to that cell. Therefore, by applying a set of different biases to an electrochemical cell, and comparing the recorded signal to a library of signals (e.g., in a look-up table) corresponding to those signals characteristic of individual known gases, it is possible to ascertain the presence of, differentiate between, and quantify the occurrence of multiple gases in the environment of the sensor. By continuously and quickly ramping the bias applied to the cell, a single electrochemical cell may rapidly differentiate between multiple gases in its environment.

In some examples, in order to provide useful gas detection information, electrochemical sensors may be relatively bulky, and thus may be ill suited for applications in which a relatively small form factor is needed. In some other examples, certain electrochemical sensors may be associated with a relatively small form factor, but may provide only limited gas detection information. For example, certain electrochemical sensors may be limited to detecting only a single target gas and/or may provide only limited (and, in some examples, inaccurate) gas concentration information.

Various aspects relate generally to an electrochemical device (e.g., an electrochemical sensor) for gas detection and/or gas concentration estimation. Some aspects more specifically relate to an electrochemical device and an associated multi-bias driving scheme that enables detection of multiple gases in an environment and/or that enables accurate concentration estimations of the multiple gases using a relatively small form factor. In some aspects, a gas detection system may include an electrochemical device and one or more components (e.g., a bias voltage driving circuit, a sensing circuit, a machine learning (ML) component, and/or similar components), coupled to the electrochemical device. The gas detection system may receive, via one or more gas apertures in the electrochemical device, a composition of one or more gases, and may apply, between one or more reference electrodes and one or more working electrodes, a set of bias voltage sub-cycles, with each bias voltage sub-cycle being associated with alternating an applied voltage between a corresponding steady voltage and a plurality of time-varying voltages centered about the corresponding steady voltage. The gas detection system may detect a current and/or a voltage while applying the set of bias voltage sub-cycles, and/or may identify a presence of one or more gases or a concentration of one or more gases based at least in part on the at least one of the detected current or the detected voltage.

Particular aspects of the subject matter described in this disclosure can be implemented to realize one or more of the following potential advantages. In some examples, the described techniques can be used to detect multiple gases and/or concentrations of multiple gases using an electrochemical device (e.g., an electrochemical sensor) that has a relatively small form factor. In some other examples, the multi-bias driving scheme and corresponding detection algorithms may enable accurate detection of multiple target gases and/or concentrations of the multiple target gases. For example, in some aspects ML may be employed to accurately classify gas types based on measurements performed on signals in the frequency domain, and ML and/or a linear-algebra-based algorithm may be used to accurately estimate the gas concentrations based on direct current (DC) signals and/or electrochemical impedance spectroscopy (EIS) signals. In this way, the aspects of the subject matter described in this disclosure may enable small form factor electrochemical sensors (sometimes referred to as miniaturized electrochemical devices) that include improved gas detection, classification, and concentration-estimate capabilities, as compared to traditional miniaturized electrochemical devices.

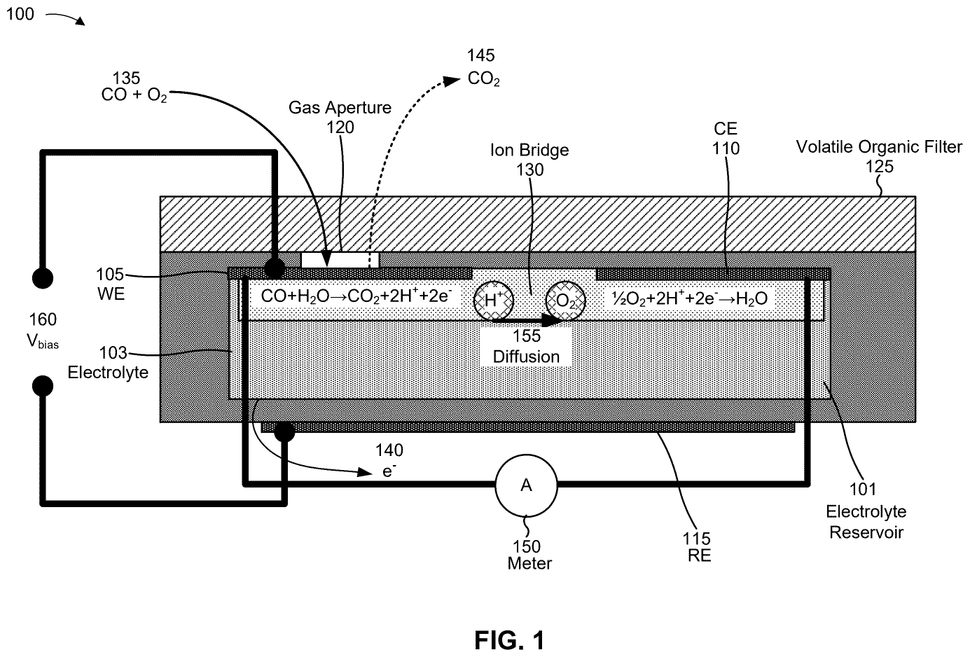

FIG. 1 is a diagram illustrating an example of an electrochemical device 100, in accordance with the present disclosure.

As shown in FIG. 1, the electrochemical device 100 may include a cavity (e.g., an electrolyte reservoir 101) that includes an electrolyte 103 in contact with a working electrode (WE) 105, a counter electrode (CE) 110, and a reference electrode (RE) 115. Additional electrodes may be added as additional WEs, CEs, and/or REs for different variants of the electrochemical device 100 for gas detection. Put another way, although only a single WE 105, CE 110, and RE 115 are shown and described for ease of description, in some aspects, the electrochemical device 100 may include multiple WEs 105, multiple CEs 110, and/or multiple REs 115. In some aspects, the RE 115 provides a means by which a reference potential may be applied to the electrochemical device 100.

The electrochemical device 100 may include a gas aperture 120 to permit diffusion into the porous WE 105 of the gas or atmosphere being sensed. In certain aspects, the gas aperture 120 may be partially or fully filled with a porous material, which may allow gas to diffuse into the WE 105 but which may block the liquid or paste-like electrolyte from exiting the cavity. Additionally, or alternatively, in some aspects the gas aperture 120 may be covered by a volatile organic filter 125. The volatile organic filter 125 may block or reduce a presence of volatile organic compounds (VOCs), such as alcohols, ketones, or solvents, among other examples, from reaching a sensing element (e.g., the WE 105 or other components of the electrochemical device 100).

In some aspects, the WE 105, CE 110, and/or RE 115 may comprise an electrically conducting material, such as carbon (among other examples), and a catalyst, such as ruthenium, copper, gold, silver, platinum, iron, ruthenium, nickel, palladium, cobalt, rhodium, iridium, osmium, vanadium, or any other suitable transition metal and/or alloys thereof. The catalyst may be selected so as to react with one or more particular gases (sometimes referred to herein as “target gases”). The WE 105, CE 110, and/or RE 115 may be partially permeable to both the electrolyte 103 and the gas to be detected so that an electrochemical reaction may occur within the body of the WE 105, CE 110, and/or RE 115. In some aspects, the electrolyte 103 may comprise an ionic material such as an acid. Additionally, or alternatively, the electrolyte 103 may be viscous (such as a gel), or may be a polymer infused with an organic or inorganic acid.

In some aspects, the electrochemical device 100 may include an ion bridge 130 (sometimes referred to as a rigid electrolyte layer) proximate to (e.g., in contact with) the WE 105 and/or the CE 110. The ion bridge 130 may be a rigid but porous polymer that is infused with an ionic material, such as sulfuric acid or phosphoric acid. In some aspects, charges may be free to move between the WE 105 and CE 110 through the ion bridge 130 to create a current signature of the target gas under the proper biasing conditions.

For example, in some aspects the electrochemical device 100 may be used to detect a presence of carbon monoxide (CO), among other gases. In such aspects, the electrochemical device 100 may include a catalyst that triggers a reaction of the target gas (e.g., CO) with water. More particularly, as indicated by reference number 135, the target gas (here CO, which may be mixed with other gases, such as oxygen (O₂), among other examples) enters the electrochemical device 100 through the volatile organic filter 125 and/or the gas aperture 120, and diffuses through the WE 105. The catalyst may trigger the target gas (e.g., CO) to react with water (H₂O), thereby releasing electrons and creating a current between the WE 105 and the CE 110, as indicated by reference number 140. The current (sometimes referred to herein as a “WE current” and/or “I_{WE}”) may be measured using a meter 150, with the current indicative of a presence of the target gas and/or a concentration of the target gas, which is described in more detail below.

For example, in aspects involving CO, the CO may react with H₂O to form carbon dioxide gas (CO₂) (which may be vented from the electrochemical device 100 via the gas aperture 120, as indicated by reference number 145), as well as free positively-charged hydrogen atoms and negatively-charged electrons (shown in FIG. 1 as CO+H₂O→CO₂+2H⁺+2e⁻). Moreover, as indicated by reference number 155, the positively-charged hydrogen atoms and/or the negatively-charged electrons may diffuse in the ion bridge 130 and/or react with the 02 that entered the electrochemical device (as described above in connection with reference number 135), thereby forming water (shown in FIG. 1 as ½O₂+2H⁺+2e⁻→H₂O).

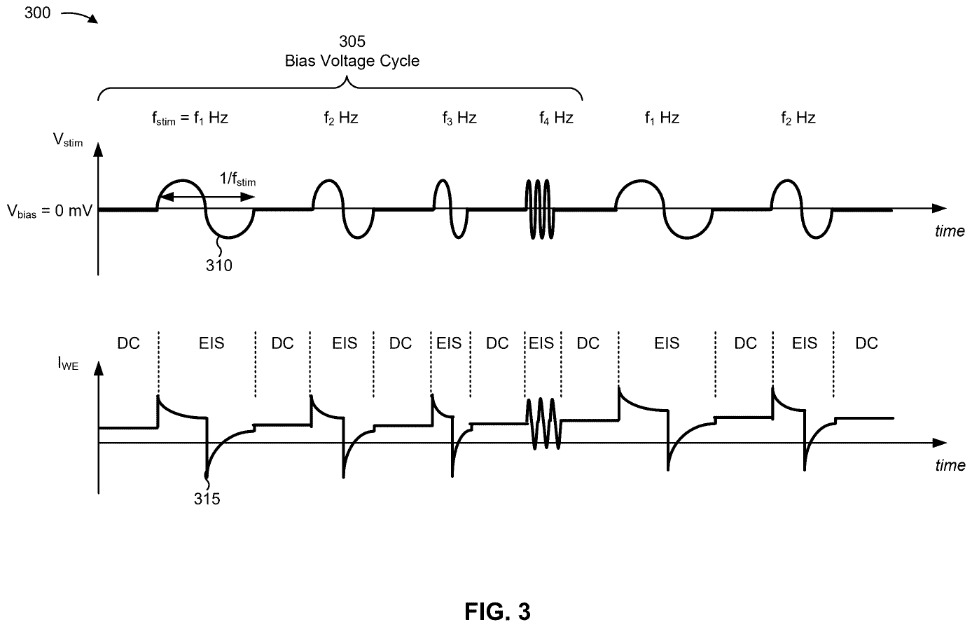

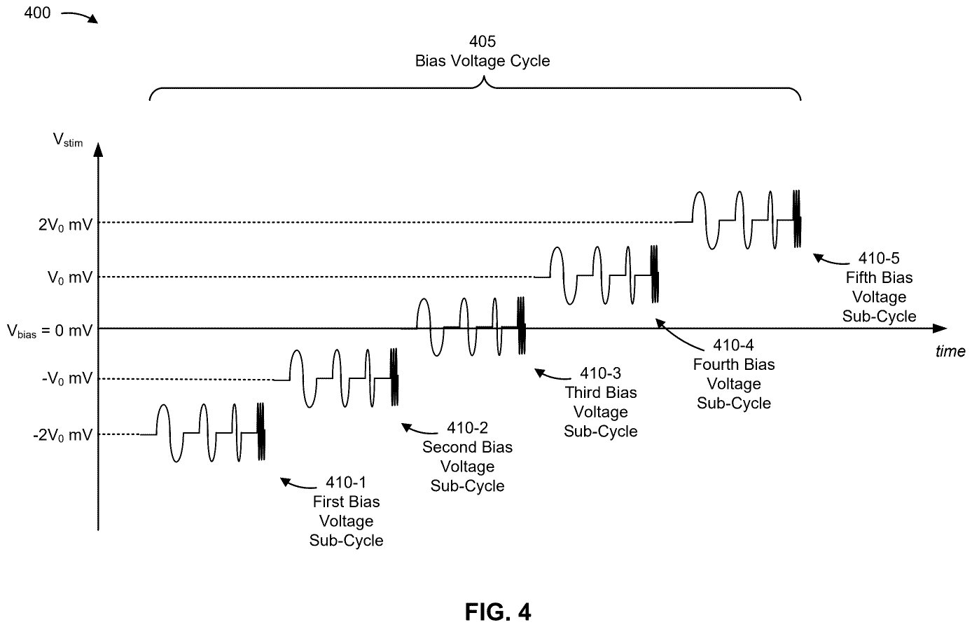

In some aspects, as indicated by reference number 160, a bias voltage (V_{bias}) may be applied between the WE 105 and the RE 115, such as for a purpose of detecting a specific gas and/or determining a concentration of a specific gas. For example, in some aspects, different gases may react and/or induce currents in the electrochemical device 100 at different bias voltages. Accordingly, a specific bias voltage may be applied to the electrochemical device 100 based on a type of gas to be detected. Moreover, in some aspects, an impedance (e.g., a resistance to alternating current (AC)) of the electrochemical device 100 may be measured, such as for a purpose of classifying a detected gas type in the electrochemical device 100. For example, in some aspects the electrochemical device 100 may utilize an EIS technique to measure the impedance of the electrochemical system over a range of frequencies, which may in turn be used to classify different gases present in the electrochemical device 100. In such aspects, a bias voltage having a certain AC waveform and/or a range of frequencies may be applied to the electrochemical device 100 to determine a presence of one or more gases in the electrochemical device 100, which is described in more detail below in connection with FIGS. 3-4.

As indicated above, FIG. 1 is provided as an example. Other examples may differ from what is described with respect to FIG. 1.

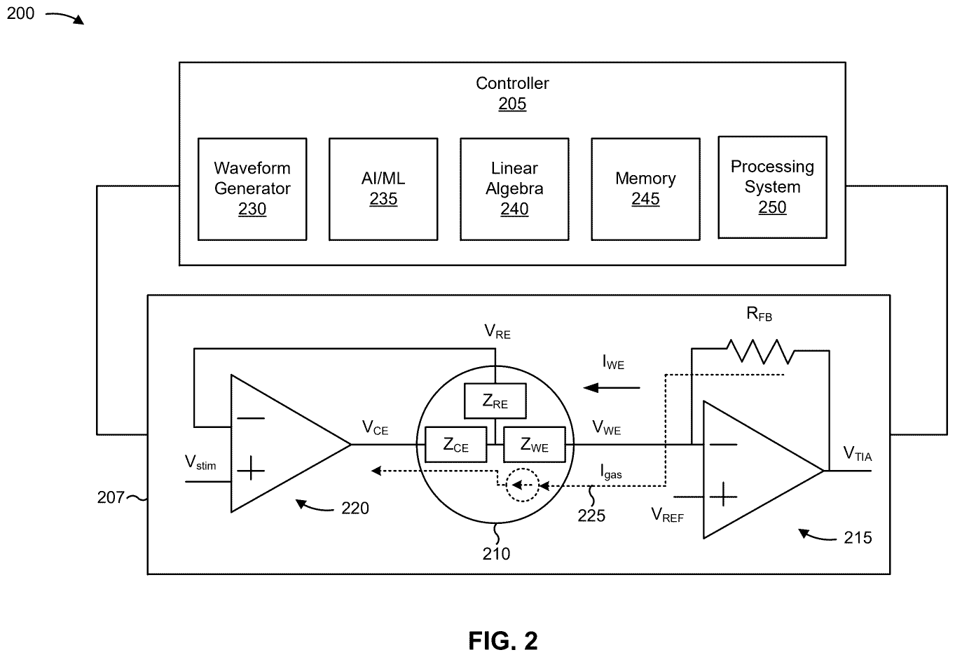

FIG. 2 is a diagram illustrating an example gas detection system 200 that implements an electrochemical device (e.g., electrochemical device 100), in accordance with the present disclosure.

As shown in FIG. 2, the gas detection system 200 may include a controller 205 (e.g., a microcontroller (MCU), a central processing unit (CPU), a field-programmable gate array (FPGA), an application specific integrated circuit (ASIC), a digital signal processor (DSP), a programmable logic controller (PLC), a system on chip (SoC), a real-time operating system (RTOS)-based controller, an embedded computer, an analog front-end (AFE) circuit, and/or a similar controller). In some aspects, the controller 205 may manage a gas detection process, such as by processing sensor data and/or setting and/or driving a bias voltage, among other examples. For example, in aspects in which an electrochemical sensor (e.g., electrochemical device 100) may generate an electrical signal that is proportional to a concentration of a specific gas in an environment, the controller 205 may process the raw data and/or may convert the raw data into a readable format, such as gas concentration (e.g., parts per million (ppm)), using calibration data, ML models, and/or linear-algebra-based algorithms, among other examples, which is described in more detail below.

In some aspects, the controller 205 may be in communication with sensing circuitry 207. The sensing circuitry 207 may include an electrochemical cell 210, which may correspond to the electrochemical device 100 described above in connection with FIG. 1. In that regard, the electrochemical cell 210 may include a WE (e.g., WE 105) having a corresponding impedance (shown in FIG. 2 as “Z_{WE}”), a CE (e.g., CE 110) having a corresponding impedance (shown in FIG. 2 as “Z_{CE}”), and/or an RE (e.g., RE 115) having a corresponding impedance (shown in FIG. 2 as “Z_{RE}”). In some aspects, the sensing circuitry 207 may be associated with a transimpedance amplifier (TIA) 215 (e.g., an operational amplifier (op-amp)) used to measure a signal (e.g., a voltage and/or a current) associated with the electrochemical cell 210, and/or a feedback loop 220 used to set a bias voltage and/or apply a stimulation waveform (sometimes referred to herein as a “stimulus voltage” (Vₛₜᵢₘ)) to the bias voltage. In some aspects, the feedback loop 220 may include an op-amp in which a stimulus voltage (e.g., Vₛₜᵢₘ, which may refer to a voltage applied to the system to perturb the electrochemical system during gas detection) is applied to a non-inverting input and in which an RE voltage (V_{RE}) (e.g., a voltage at the RE 115, which may be used to monitor the potential difference between the WE and the RE) is applied to an inverting input, with an output voltage thereof corresponding to a CE voltage (V_{CE}) (e.g., a voltage at the CE 110, which in some aspects may be adjusted to maintain a desired voltage at the WE 105).

Moreover, the TIA 215 may include an op-amp in which a reference voltage (V_{REF}) (e.g., a voltage at the RE 115 relative to ground or some baseline voltage, which may serve as a stable point of reference to control the voltage at the WE) applied to a non-inverting input and in which a WE voltage (V_{WE}) (e.g., a voltage applied or measured at the WE 105 relative to the RE 115) is applied to an inverting input, with a resulting output voltage being a TIA output voltage (V_{TIA}) (e.g., a voltage which converts the current generated by the WE due to a chemical reaction at the electrochemical cell 210 into a measurable voltage). Moreover, in some aspects the TIA 215 may be associated with a feedback resistor (R_{FB}), which may set the gain of the TIA 215 by determining how much current (sometimes referred to as I, which may correspond to I_{WE}) is converted into voltage (e.g., V=I×R_{FB}). In this way, the gas detection system 200 may apply various bias voltages (e.g., V_{bias}) and/or stimulus waveforms (e.g., Vₛₜᵢₘ) to the electrochemical cell and/or may measure a resulting voltage (e.g., V_{TIA}) and/or an associated current (e.g., I_{WE}) to detect a presence of one or more gases and/or a concentration of each gas.

For example, as indicated by reference number 225, a target gas may enter the electrochemical cell 210, such as via one or more gas apertures (e.g., gas aperture 120). The target gas may react with the electrochemical cell 210 in a similar manner as described above in connection with the electrochemical device 100, thereby creating a current in the electrochemical cell 210 (shown in FIG. 2 as “I_{gas}”). The controller 205 of the gas detection system 200 may cause one or more bias voltages to be applied to the electrochemical cell 210 and/or may cause one or more stimulus waveforms to be applied to the bias voltages while the target gas is present in the electrochemical cell 210, such as by using a waveform generator component 230 of the controller 205 (which may be in communication with the feedback loop 220 and/or op-amp thereof). While applying the one or more bias voltages to electrochemical cell 210 and/or the one or more stimulus waveforms to the bias voltages, the controller 205 of the gas detection system 200 may detect and/or measure V_{TIA} and/or a current associated with V_{TIA} (e.g., I_{WE}, such as by using meter 150 and/or a similar meter), such as for a purpose of detecting a presence of one or more target gases and/or a concentration of each gas. In some other aspects, different signals (e.g., currents and/or voltages) associated with the electrochemical cell 210 may be measured and/or detected for a purpose of detecting one or more gases and/or calculating a concentration of one or more gases, without departing from the scope of the disclosure.

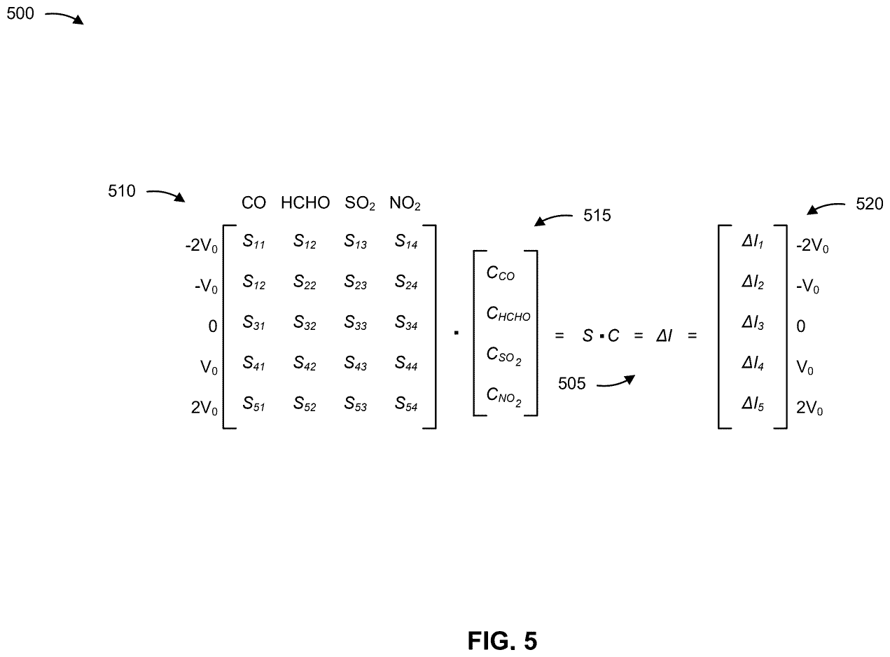

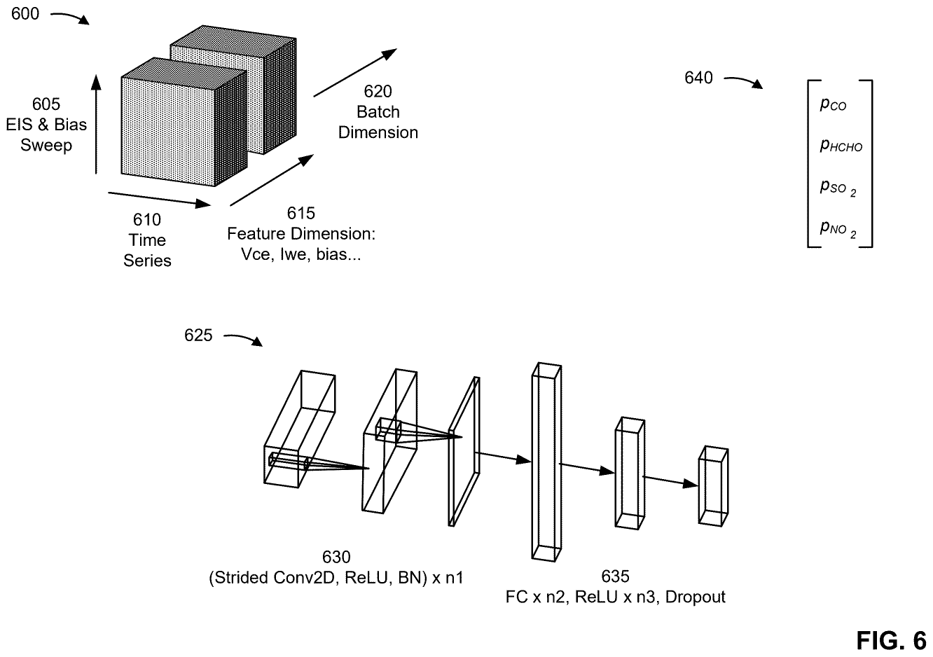

In some aspects, the controller 205 may detect a presence of a target gas and/or a concentration thereof using one or more of an artificial intelligence (AI)/ML component 235, a linear algebra component 240, and/or a similar component. More particularly, in some aspects, a presence and/or concentration of one or more gases may be determined, at least in part, using an AI program (sometimes referred to herein as an “AI/ML model”) associated with the AI/ML component 235 and/or in communication with the AI/ML component 235, such as a program that includes an ML model and/or an artificial neural network (ANN) model. The AI/ML model may be deployed at the controller 205 and/or one or more other devices in communication with the controller 205 and/or the electrochemical cell 210 and/or the sensing circuitry 207, such as at a remote server and/or cloud-based computing environment, among other examples. In some examples, the AI/ML model (or an instance of the AI/ML model) may be deployed at multiple devices (for example, a first portion of the AI/ML model may be deployed at the controller 205 and a second portion of the AI/ML model may be deployed at a server, a cloud-based computing environment, and/or other device in communication with the controller 205). In other examples, a first AI/ML model may be deployed at the controller 205 and a second AI/ML model may be deployed in a server, a cloud-based computing device, and/or other device in communication with the controller 205. The AI/ML model(s) may be configured to enhance various aspects of the gas detection system 200. For example, the AI/ML model(s) may be trained to identify patterns or relationships in data corresponding to the gas detection system 200, such as patterns or relationships associated with a presence of one or more target gases and/or a concentration of one or more target gases, among other examples. Additional aspects of the AI/ML component 235 and/or the linear algebra component 240 are described in more detail below in connection with FIGS. 5 and 6.

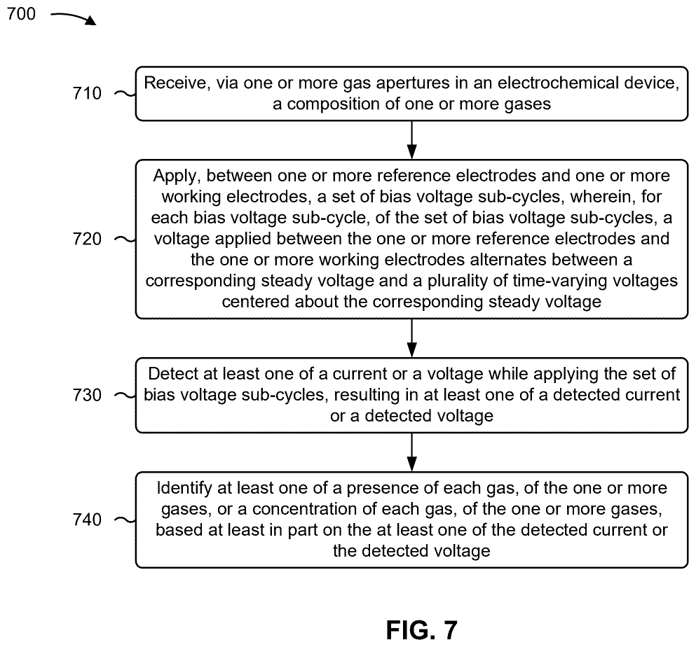

The electrochemical device 100, the gas detection system 200, or any other component(s) of FIG. 1 and/or FIG. 2 may implement one or more techniques or perform one or more operations associated with detecting a gas using a multi-bias scheme, as described in more detail elsewhere herein. For example, the electrochemical device 100 and/or the gas detection system 200 may perform or direct operations of, for example, process 700 of FIG. 7, or other processes as described herein (alone or in conjunction with one or more other processors). Additionally, or alternatively, in some aspects, memory 245 of the controller 205 may store data and program code (or instructions) for the controller 205, the sensing circuitry 207, and/or the electrochemical cell 210, among other examples. In some examples, the memory 245 of the controller 205 may include a non-transitory computer-readable medium storing a set of instructions. For example, the set of instructions, when executed by one or more processors (for example, processing system 250) of the controller 205, may cause the one or more processors to perform process 700 of FIG. 7, or other processes as described herein. In some examples, executing instructions may include running the instructions, converting the instructions, compiling the instructions, and/or interpreting the instructions, among other examples.

In some aspects, the gas detection system 200 may include means for receiving, via one or more gas apertures in an electrochemical device, a composition of one or more gases; means for applying, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage; means for detecting at least one of a current or a voltage while applying the set of bias voltage sub-cycles, resulting in at least one of a detected current or a detected voltage; and means for identifying at least one of a presence of each gas, of the one or more gases, or a concentration of each gas, of the one or more gases, based at least in part on the at least one of the detected current or the detected voltage. The means for the gas detection system to perform operations described herein may include, for example, one or more of the electrochemical device 100, the meter 150, the controller 205, the sensing circuitry 207, the electrochemical cell 210, the TIA 215, the feedback loop 220, the waveform generator component 230, the AI/ML component 235, the linear algebra component 240, the memory 245, and/or the processing system 250, among other examples.

As indicated above, FIG. 2 is provided as an example. Other examples may differ from what is described with respect to FIG. 2.

FIG. 3 is a diagram illustrating an example 300 associated with single bias driving scheme for an electrochemical gas sensor, in accordance with the present disclosure.

The example 300 shown in FIG. 3 may be a bias scheme implemented by a gas detection system, such as the gas detection system 200 or a similar gas detection system. As shown in FIG. 3, the single bias driving scheme may include applying a bias voltage cycle 305 (sometimes referred to herein as a “driving cycle”) to an electrochemical device, such as the electrochemical device 100 and/or the electrochemical cell 210. For example, the bias voltage cycle 305 may be applied between one or more WEs (e.g., one or more WEs 105) and one or more REs (e.g., one or more REs 115). As indicated by the voltage curve 310, which shows a stimulus voltage (e.g., Vₛₜᵢₘ) versus time associated with the bias voltage cycle 305, the bias voltage cycle 305 may include applying a voltage (e.g., between the one or more REs and the one or more WEs) that alternates between a steady voltage (e.g., a DC voltage, which may correspond to V_{bias}) and a plurality of time-varying voltages (e.g., an AC voltage) centered about the steady voltage. For example, in this aspect, the steady voltage may correspond to V_{bias}, which may be 0 mV. Moreover, the plurality of time-varying voltages may have a sine-wave waveform centered about the steady voltage (0 mV in this example) that may alternate according to a stimulus frequency (fₛₜᵢₘ), and thus a wavelength associated with each time-varying voltage may be equal to 1/fₛₜᵢₘ, as shown in connection with the first time-varying voltage of the voltage curve 310.

In some aspects, within each bias voltage cycle 305, a stimulus frequency of a given time-varying voltage may increase with respect to a stimulus frequency of a previous time-varying voltage. Put another way, the gas detection system may sweep a stimulus frequency within the bias voltage cycle 305. For example, in this aspect, a stimulus frequency (e.g., fₛₜᵢₘ) for a first time-varying voltage may be equal to f₁ hertz (Hz), a stimulus frequency for a second time-varying voltage may be equal to f₂ Hz, a stimulus frequency for a third time-varying voltage may be equal to f₃ Hz, and/or a stimulus frequency for a fourth time-varying voltage may be equal to f₄ Hz. Accordingly, with a given bias voltage cycle 305, a steady voltage (e.g., a DC voltage, such as 0 mV) may be applied to an electrochemical device during a first portion of the bias voltage cycle 305, a time-varying voltage (e.g., an AC voltage) centered at the steady voltage (e.g., 0 mV) and having a first stimulus frequency (e.g., fₛₜᵢₘ=f₁ Hz) may be applied during a second portion of the bias voltage cycle 305, the steady voltage (e.g., 0 mV) may be applied during a third portion of the bias voltage cycle 305, a second time-varying voltage centered at the steady voltage and having a second stimulus frequency (e.g., fₛₜᵢₘ=f₂ Hz) may be applied during a fourth portion of the bias voltage cycle 305, and so forth, as shown in FIG. 3. After all time-varying voltages have been applied (e.g., after all four time-varying voltages associated with stimulus frequencies of f₁ Hz through f₄ Hz in the example shown in FIG. 3 have been applied), the bias voltage cycle 305 may be repeated, such that the steady voltage (e.g., 0 mV) may be applied next, following by the time-varying voltage having the first stimulus frequency (e.g., fₛₜᵢₘ=f₁ Hz), and so forth.

Although four time-varying voltages (and thus four instances of the steady voltage) are shown in connection with the bias voltage cycle 305, in some other aspects, more or fewer time-varying voltages (and thus more or fewer instances of the steady voltage) may be implemented without departing from the scope of the disclosure. Additionally, or alternatively, in some aspects, the time-varying voltages may be associated with stimulus frequencies between 1 Hz and 1 megahertz (MHz), while, in some other aspects, the time-varying voltages may be associated with stimulus frequencies higher than 1 MHz (e.g., 10 MHz or the like) or lower than one Hz (e.g., 0.1 Hz, 0.01 Hz, or the like) without departing from the scope of the disclosure. Moreover, each time-varying voltage may include one or more periods. For example, in the aspect shown in FIG. 3, the first, second, and third time-varying voltages (e.g., the time-varying voltages associated with a stimulus frequency of f₁ Hz, f₂ Hz, and f₃ Hz, respectively) are associated with a single period, while the fourth time-varying voltage (e.g., the time-varying voltage associated with a stimulus frequency of f₄ Hz) is associated with multiple periods (e.g., three periods). However, in some other aspects, the first, second, and third time-varying voltages may be associated with more than one period and/or the fourth time-varying voltage may be associated with one, two, or more than three periods without departing from the scope of the disclosure.

As indicated by the current curve 315, which shows a current at the WE (e.g., I_{WE}) versus time, current at a WE (e.g., WE 105) may be measured during application of the bias voltage cycle 305, such as for a purpose of detecting one or more gases at the electrochemical device and/or determining a concentration of the one or more gases at the electrochemical device. As indicated by the portions of the current curve labeled “DC,” a response of the electrochemical device (e.g., a current measured at the WE) when the steady voltage (e.g., 0 mV) is applied to the electrochemical device may be relatively constant. In some aspects, this response may be used to calculate a concentration of a gas at the electrochemical sensor. For example, higher concentrations of a given gas may result in higher currents at the WE, and thus a measurement of a steady state current in response to the steady voltage may be used by the gas detection system to estimate a concentration (e.g., ppm) of the gas at the electrochemical device.

Moreover, as indicated by the portions of the current curve labeled “EIS,” a response of the electrochemical device (e.g., a current measured at the WE) when the time-varying voltages (e.g., the AC voltages that alternate according to fₛₜᵢₘ) are applied to the electrochemical device may vary with time. In some aspects, this response may be used to identify one or more gases at the electrochemical sensor. For example, the current measured during application of the time-varying voltages to the electrochemical device may be used in connection with an EIS scheme that measures impedance (e.g., resistance to AC) of the electrochemical device over a range of frequencies (e.g., f₁ Hz to f₄ Hz in this example) and/or which may determine whether one or more target gases are present at the electrochemical device based at least in part on the measured impedance. In this way, by employing a bias voltage cycle 305 that applies a voltage that alternates between a steady voltage (e.g., a DC voltage) and a plurality of time-varying voltages (e.g., an AC voltage) centered about the steady voltage, the gas detection system may be capable of detecting a presence of one or more target gases and/or a concentration of one or more target gases.

In some aspects, using a single, repeating bias voltage cycle (such as the bias voltage cycle 305 shown in FIG. 3) to detect gases and/or calculate a concentration of gases may be sufficient for certain gases but insufficient for other gases. For example, the bias voltage cycle 305 may be configured and/or tuned for detecting CO and/or for estimating a concentration of CO, but may not be capable of accurately detecting other gases, such as formaldehyde (HCHO), sulfur dioxide (SO₂), and/or nitrogen dioxide (NO₂), among other examples. Accordingly, in some aspects, a gas detection system may sweep a bias voltage across multiple bias voltages and/or may apply a bias voltage cycle (e.g., similar to bias voltage cycle 305) at each bias voltage, such as for a purpose of detecting multiple constituent gases in a composition of gases and/or a concentration of each of multiple gases in a composition of gases, among other examples. Aspects of sweeping a bias voltage across multiple bias voltages and/or applying a bias voltage cycle (e.g., similar to bias voltage cycle 305) at each bias voltage are described in more detail below in connection with FIGS. 4-6.

As indicated above, FIG. 3 is provided as an example. Other examples may differ from what is described with respect to FIG. 3.

FIG. 4 is a diagram illustrating an example 400 associated with a multi-bias driving scheme for an electrochemical gas sensor, in accordance with the present disclosure.

The example 400 shown in FIG. 4 may be a bias scheme implemented by a gas detection system, such as the gas detection system 200 or a similar gas detection system. As shown in FIG. 4, the multi-bias driving scheme may include applying a bias voltage cycle 405 (e.g., driving cycle) to an electrochemical device, such as the electrochemical device 100 and/or the electrochemical cell 210. For example, the bias voltage cycle 405 may be applied between one or more WEs (e.g., one or more WEs 105) and one or more REs (e.g., one or more REs 115) of an electrochemical device.

As shown in FIG. 4, in this aspect the bias voltage cycle 405 may comprise multiple bias voltage sub-cycles 410 (shown in FIG. 4 as a first bias voltage sub-cycle 410-1 through a fifth bias voltage sub-cycle 410-5, but which may include more or fewer bias voltage sub-cycles 410 in other aspects). Each bias voltage sub-cycle 410 may be similar to the bias voltage cycle 305 described above in connection with FIG. 3. However, in this aspect the gas detection system may sweep a bias voltage (e.g., V_{bias}) throughout the bias voltage cycle 405, such that each bias voltage sub-cycle 410 is centered about a different steady voltage (e.g., a different bias voltage). For example, in this aspect, the first bias voltage sub-cycle 410-1 is centered about a V_{bias} of −2V₀ mV, the second bias voltage sub-cycle 410-2 is centered about a V_{bias} of −V₀ mV, the third bias voltage sub-cycle 410-3 is centered about a V_{bias} of 0 mV, the fourth bias voltage sub-cycle 410-4 is centered about a V_{bias} of V₀ mV, and the fifth bias voltage sub-cycle 410-5 is centered about a V_{bias} of 2V₀ mV. In some other aspects, the bias voltage sub-cycles 410 may be centered about bias voltages that differ from those shown in FIG. 4, without departing from the scope of disclosure.

In that regard, applying a voltage at an electrochemical device according to the bias voltage cycle 405 may include applying (e.g., between one or more REs and one or more WEs) a set of bias voltage sub-cycles (e.g., the first bias voltage sub-cycle 410-1 through the fifth bias voltage sub-cycle 410-5), such that for each bias voltage sub-cycle 410, an applied voltage alternates between a corresponding steady voltage (e.g., the corresponding V_{bias} for that bias voltage sub-cycle 410, such as one of −2V₀ mV, −V₀ mV, 0 mV, V₀ mV, or 2V₀ mV in the example shown in FIG. 4) and a plurality of time-varying voltages (e.g., the sine-wave voltage waveforms shown in FIG. 4) centered about the corresponding steady voltage. Moreover, and in a similar manner as described above in connection with the bias voltage cycle 305, within each bias voltage sub-cycle 410, a stimulus frequency (e.g., fₛₜᵢₘ) of a given time-varying voltage may increase with respect to a stimulus frequency of a previous time-varying voltage within that bias voltage sub-cycle 410. Put another way, the gas detection system may sweep a stimulus frequency within the bias voltage sub-cycle 410.

Accordingly, with a given bias voltage sub-cycle 410, a steady voltage (e.g., a DC voltage, such as one of −2V₀ mV, −V₀ mV, 0 mV, V₀ mV, or 2V₀ mV, among other examples) may be applied to an electrochemical device during a first portion of the bias voltage sub-cycle 410, a time-varying voltage (e.g., an AC voltage) centered at the steady voltage (e.g., the one of the −2V₀ mV, −V₀ mV, 0 mV, V₀ mV, or 2V₀ mV) and having a first stimulus frequency (e.g., fₛₜᵢₘ=f₁ Hz) may be applied during a second portion of the bias voltage sub-cycle 410, the steady voltage (e.g., the one of the −2V₀ mV, −V₀ mV, 0 mV, V₀ mV, or 2V₀ mV) may be applied during a third portion of the bias voltage sub-cycle 410, a second time-varying voltage centered at the steady voltage and having a second stimulus frequency (e.g., fₛₜᵢₘ=f₂ Hz) may be applied during a fourth portion of the bias voltage sub-cycle 410, and so forth. Put another way, in aspects in which the time-varying voltages of each bias voltage sub-cycle 410 are associated with AC voltages (such as the sine-wave voltage waveform shown in FIG. 4, among other examples described in more detail below), for each bias voltage sub-cycle 410, a frequency of each AC voltage differs from frequencies of all other AC voltages. In some other aspects, such as in aspects in which the time-varying voltages of each bias voltage sub-cycle 410 are associated with a voltage having various levels but not necessarily an AC voltage (such as a step-wave voltage waveform, a sawtooth-wave voltage waveform, or a ramp-wave voltage waveform, among other examples described in more detail below), for each bias voltage sub-cycle 410, a duration associated with each voltage level may differ from durations associated with all other time-varying voltages. For example, for aspects involving a step-wave voltage waveform, a duration spent at each voltage step in the first time-varying voltage of a given bias voltage sub-cycle 410 may differ from a duration spent at each voltage step in the second time-varying voltage of a given bias voltage sub-cycle 410, and so forth.

Moreover, after all time-varying voltages have been applied for a given bias voltage sub-cycle 410 (e.g., after all four time-varying voltages associated with stimulus frequencies of f₁ Hz through f₄ Hz in the example shown in FIG. 4 have been applied), a bias voltage may be changed (e.g., stepped up) and a next bias voltage sub-cycle 410 may be applied. For example, following the first bias-voltage sub-cycle 410-1, a bias voltage may be stepped up to −V₀ mV from −2V₀ mV, and the second bias voltage sub-cycle 410-2 may be applied, which may be similar to the first bias voltage sub-cycle 410-1 but which is centered about a steady voltage of −V₀ mV. Similarly, following the second bias voltage sub-cycle 410-2, the third bias voltage sub-cycle 410-3, the fourth bias voltage sub-cycle 410-4, and the fifth bias voltage sub-cycle 410-5 may be applied, centered around bias voltages of 0 mV, V₀ mV, and 2V₀ mV, respectively. Moreover, in some aspects, once all bias voltage sub-cycles 410 have been applied, the bias voltage cycle 405 may be repeated, such that the bias voltage may be stepped down to −2V₀ mV, the first bias voltage sub-cycle 410-1 may be applied next, followed by second bias voltage sub-cycle 410-2, and so forth.

Although, five bias voltage sub-cycles 410 are shown in connection with the bias voltage cycle 405, in some other aspects, more or fewer bias voltage sub-cycles 410 may be implemented without departing from the scope of the disclosure. Moreover, although each bias voltage sub-cycle 410 includes four time-varying voltages (and thus four instances of the steady voltage), in some other aspects, more or fewer time-varying voltages (and thus more or fewer instances of the steady voltage) may be implemented at each bias voltage sub-cycle 410 without departing from the scope of the disclosure. Additionally, or alternatively, in some aspects, the time-varying voltages may be associated with stimulus frequencies between 1 Hz and 1 MHz, while, in some other aspects, the time-varying voltages may be associated with stimulus frequencies higher than 1 MHz (e.g., 10 MHz or the like) or lower than one Hz (e.g., 0.1 Hz, 0.01 Hz, or the like) without departing from the scope of the disclosure. Moreover, each time-varying voltage of each bias voltage sub-cycle 410 may include one or more periods. For example, in the aspect shown in FIG. 4, the first, second, and third time-varying voltages of each bias voltage sub-cycle 410 are shown as including a single period, while the fourth time-varying voltage is shown as including multiple periods (e.g., three periods). However, in some other aspects, the first, second, and third time-varying voltages of each bias voltage sub-cycle 410 may be associated with more than one period and/or the fourth time-varying voltage of each bias voltage sub-cycle 410 may be associated with one, two, or more than three periods without departing from the scope of the disclosure.

Moreover, although the time-varying voltages at each bias voltage sub-cycle 410 are shown and described as a sine-wave voltage waveform, in some other aspects the time-varying voltages at each bias voltage sub-cycle 410 may be associated with a different time-varying voltage waveform (e.g., a different AC voltage and/or non-AC, yet still time-varying, voltage waveform). For example, in some aspects, the time-varying voltages may be associated with square-wave voltage waveforms, sine-wave voltage waveforms, sawtooth-wave voltage waveforms, triangular-wave voltage waveforms, step-wave voltage waveforms, pulse-wave voltage waveforms, ramp-wave voltage waveforms, exponential-wave voltage waveforms, and/or modulated-wave voltage waveforms, among other examples.

Moreover, in some aspects, a gas detection system implementing the bias voltage cycle 405 shown in FIG. 4 or a similar bias voltage cycle may apply the bias voltage sub-cycles 410 by either controlling a current at the electrochemical device or a voltage at the electrochemical device. For example, in some aspects the waveform generator component 230 of the gas detection system 200 and/or a similar component may include or otherwise be associated with an electronic instrument used to regulate a voltage between electrodes in the electrochemical device (e.g., a voltage between the WE and RE), such as a potentiostat or similar instrument, and/or an electronic instrument used to regulate a current between electrodes in the electrochemical device (e.g., a current between the WE and CE), such as a galvanostat or similar instrument. In such aspects, the gas detection system may apply the set of bias voltage sub-cycles 410 by using a potentiostat to control a voltage between the one or more REs and the one or more WEs and/or by using a galvanostat to control a current between the one or more WEs and the one or more CEs.

By implementing multiple bias voltage sub-cycles 410 and/or by sweeping bias voltages between subsequent bias voltage sub-cycles 410 as well as sweeping time-varying voltages within each bias voltage sub-cycle 410, a gas detection system may be capable of more accurately detecting multiple gases within a composition of gases and/or a concentration of each gas within a composition of gases. Aspects of detecting multiple gases and/or the concentrations thereof using multiple bias voltage sub-cycles 410 are described in more detail below in connection with FIGS. 5 and 6.

As indicated above, FIG. 4 is provided as an example. Other examples may differ from what is described with respect to FIG. 4.

FIG. 5 is a diagram illustrating an example 500 associated with calculating concentrations of gases using a multi-bias driving scheme for an electrochemical gas sensor, in accordance with the present disclosure.

In some aspects, an electrochemical device's response to a presence of a target gas may be proportional to a concentration of the target gas at the electrochemical device (e.g., the electrochemical device 100 and/or the electrochemical cell 210). For example, in the context of an electrochemical device that is configured to detect a presence of CO, a change in a current at the WE (e.g., WE 105), sometimes referred to herein as “ΔI_{WE},” may be proportional to a concentration of CO at the electrochemical device. More particularly, when no particles of a target gas (e.g., CO) are present at the electrochemical device (e.g., when normal, uncontaminated air is at the electrochemical device), a baseline current (sometimes referred to herein as “I_{baseline}”) may be sensed at the electrochemical device (e.g., at the WE of the electrochemical device). For example, I_{baseline} may be relatively small, such as very near 0 nanoamps (nA).

In some aspects, the current sensed at the electrochemical device (e.g., I_{WE}) may increase as the electrochemical device is exposed to a target gas (e.g., CO in this example) by an amount proportional to the concentration of the target gas at the electrochemical device. For example, I_{WE} may be equal to I_{baseline} when no CO is present at the electrochemical device, I_{WE} may be greater than I_{baseline} when a first concentration (e.g., 30 ppm) of CO is present at the electrochemical device (e.g., I_{we,30ppm}>I_{baseline}), I_{WE} may be even greater than I_{baseline} when a higher concentration (e.g., 70 ppm) of CO is present at the electrochemical device (e.g., I_{we,70ppm}>I_{we,30ppm}>I_{baseline}), I_{WE} may be even greater than I_{baseline} when an even higher concentration (e.g., 100 ppm) of CO is present at the electrochemical device (e.g., I_{we,100ppm}>I_{we,70ppm}>I_{we,30ppm}>I_{baseline}), and so forth. In that regard, using a known sensitivity of the electrochemical device to a target gas (sometimes referred to herein as “S_{gas},” such as S_{CO} for CO), the concentration of the target gas (sometimes referred to herein as “C_{gas},” such as C_{CO} for CO) may be estimated using a difference of the I_{WE} as compared to I_{baseline} (sometimes referred to as “ΔI,” which may be defined as I_{WE}−I_{baseline}). More particularly, S_{gas}×C_{gas}=ΔI defI_{WE}−I_{baseline}. In that regard, a concentration of a single gas at the electrochemical device may be determined based on the following expression:

$${C}_{gas} = \frac{Δ I}{{S}_{gas}} .$$

In some aspects, the electrochemical device may have different sensitivities to different gases. For example, a sensitivity of the electrochemical device to CO may be different than a sensitivity of the electrochemical device to HCHO, SO₂, and/or NO₂, among other examples. This may present a challenge when using a single electrochemical device to measure concentrations of multiple target gases, particularly since the electrochemical device's response to mixed gases (e.g., a composition of two or more gases) may be additive. For example, in aspects involving an electrochemical device used to detect concentrations of four target gases, such as CO, HCHO, SO₂, and NO₂, the electrochemical device's response to the target gases may be expressed as S_{CO}×C_{CO}+S_{HCHO}×C_{HCHO}+S_{SO}_{₂}×C_{SO}_{₂}+S_{NO}_{₂}×C_{NO}_{₂}=ΔI. Accordingly, performing only one measurement (e.g., ΔI) and/or using only one known sensitivity of the electrochemical device to a target gas (e.g., one S_{gas}) may be insufficient to determine a concentration of multiple target gases (e.g., CO, HCHO, SO₂, and/or NO₂, among other examples).

Accordingly, in some aspects, a gas detection system (e.g., the linear algebra component 240 of the gas detection system 200) may use a linear-algebra-based algorithm to estimate the concentration of each gas in a composition of multiple gases. More particularly, by sweeping the bias voltage (e.g., steady voltage) across multiple different voltages during a bias voltage cycle (such as by sweeping the bias voltage across −2V₀ mV, −V₀ mV, 0 mV, V₀ mV, and 2V₀ mV as described above in connection with the bias voltage cycle 405 of example 400), by measuring the electrochemical device's response to each bias voltage, and/or by using a sensitivity matrix associated with the electrochemical device's sensitivity to each of multiple target gases, a gas detection system may be able to accurately determine a concentration of multiple constituent gases in a composition of gases.

More particularly, as shown in example 500, and as indicated by reference number 505, a change in current at an electrochemical device, such as a change in a current at a WE of an electrochemical device (shown in FIG. 5 simply as “ΔI”), may be equal to a sensitivity of an electrochemical device to one or more target gases (shown in FIG. 5 simply as “S”) multiplied by a concentration of the one or more target gases (shown in FIG. 5 simply as “C”). For a gas detection system associated with multiple target gases, this expression may be represented using a system of equations including a sensitivity matrix 510, a concentration matrix 515, and a current matrix 520. The sensitivity matrix 510 may include known sensitivities of the electrochemical device to the various target gases at various bias voltages. For example, the first row of the sensitivity matrix 510 may include values corresponding to the sensitivity of the electrochemical device to the presence of CO (shown as S₁₁), HCHO (shown as S₁₂), SO₂ (shown as S₁₃), and NO₂ (shown as S₁₄) when a bias voltage is equal to −2V₀ mV. Similarly, the second row of the sensitivity matrix 510 may include values corresponding to the sensitivity of the electrochemical device to the presence of CO, HCHO, SO₂, and NO₂ (shown as S₂₁ through S₂₄, respectively) when a bias voltage is equal to −V₀ mV. The third row of the sensitivity matrix 510 may include values corresponding to the sensitivity of the electrochemical device to the presence of CO, HCHO, SO₂, and NO₂ (shown as S₃₁ through S₃₄, respectively) when a bias voltage is equal to 0 mV. Moreover, the fourth row of the sensitivity matrix 510 may include values corresponding to the sensitivity of the electrochemical device to the presence of CO, HCHO, SO₂, and NO₂ (shown as S₄₁ through S₄₄, respectively) when a bias voltage is equal to V₀ mV. And the fifth row of the sensitivity matrix 510 may include values corresponding to the sensitivity of the electrochemical device to the presence of CO, HCHO, SO₂, and NO₂ (shown as S₅₁ through S₅₄, respectively) when a bias voltage is equal to 2V₀ mV.

In this way, the unknown concentrations of the target gases (e.g., C_{CO}, C_{HCHO}, C_{SO}_{₂}, and C_{NO}_{₂}, as shown in the concentration matrix 515) may be determined by measuring the system's response (e.g., a change in current) at the various bias voltages, as shown in the current matrix 520 (e.g., by measuring ΔI₁ at −2V₀ mV, ΔI₂ at −V₀ mV, ΔI₃ at 0 mV, ΔI₄ at V₀ mV, and ΔI₅ at 2V₀ mV), and by solving the system of equations shown in example 500 for the unknown variables included in the concentration matrix 515. Moreover, as explained above in connection with FIGS. 3 and 4, in some aspects the measurements used for the concentration matrix (e.g., ΔI₁ through ΔI₅) may be measurements performed when a corresponding steady voltage (e.g., DC voltage) is being applied for a given bias voltage subs-cycle. Put another way, in some aspects, using a linear-algebra-based algorithm to estimate the concentration of each gas (e.g., a linear-algebra-based algorithm associated with the matrices depicted in FIG. 5) may be based at least in part on a detected current and/or voltage while steady voltages are applied between one or more REs and one or more WEs of an electrochemical device. However, in some other aspects, using the linear-algebra-based algorithm to estimate the concentration of each gas may be based at least in part on a detected current and/or voltage while time-varying voltages are applied between the one or more REs and the one or more WEs, without departing from the scope of the disclosure.

Additionally, or alternatively, although the above example has been described in connection with detected currents at the WE (e.g., I_{WE} and/or ΔIdefI_{WE}−I_{baseline}), in some other aspects a different detected current and/or voltage of a electrochemical device may be used to determine a system's response to a presence of a detected gas and/or to determine a concentration of one or more target gases. For example, in some aspects, one or more of the currents and/or voltages described above in connection with FIG. 2 (e.g., V_{TIA}, V_{REF}, I_{WE}, V_{WE}, V_{RE}, V_{CE}, and/or Vₛₜᵢₘ) may be measured and/or detected during application of a bias voltage cycle (e.g., bias voltage cycle 405), such as for a purpose of determining a concentration of one or more target gases. More particularly, in some aspects, a detected current and/or the detected voltage used to determine a concentration of one or more target gases may be a current detected between one or more WEs and one or more CEs (e.g., I_{WE}), a voltage detected between the one or more WEs and one or more REs (e.g., V_{WE}), a voltage detected between the one or more CEs and the one or more REs (e.g., V_{CE}), and/or a voltage detected between the one or more CEs and the one or more WEs (e.g., V_{CE}−V_{WE}, sometimes referred to as a “cell voltage”), among other examples.

Moreover, although the example shown and described in connection with FIG. 5 is associated with a gas detection system using a linear-algebra-based algorithm to estimate the concentration of each target gas, in some other aspects a gas detection system may use an ML model to calculate the concentration of each target gas. For example, a gas detection system (e.g., gas detection system 200) may use an AI/ML component (e.g., AI/ML component 235) and/or an associated ML model to calculate the concentration of each target gas, which may be performed in a similar manner to the aspects described below in connection with FIG. 6.

As indicated above, FIG. 5 is provided as an example. Other examples may differ from what is described with respect to FIG. 5.

FIG. 6 is a diagram illustrating examples associated with detecting presence of gases using a multi-bias driving scheme for an electrochemical gas sensor, in accordance with the present disclosure.

In some aspects, a gas detection system (e.g., gas detection system 200 and/or the AI/ML component 235 of the gas detection system 200) may identify a presence of each target gas using an ML model to calculate probabilities that one or more target gases are present at the electrochemical device. Additionally, or alternatively, in some aspects, such as aspects in which EIS techniques are used to detect the presence of one or more target gases, the gas detection system may calculate probabilities that one or more gases are present based at least in part on a detected current and/or voltage while time-varying voltages are applied at the electrochemical device, as described above in connection with FIGS. 3 and 4.

As indicated by reference number 600 in FIG. 6, a setup of a tensor (e.g., a multi-dimensional array of data compatible for use in an ML model, such as a deep-learning network) associated with an ML model that may be used to calculate probabilities that one or more target gases are present at the electrochemical device and/or concentrations of one or more target gases. As indicated by reference number 600, a tensor may include multiple dimensions, such as an EIS and/or bias sweep dimension 605 (e.g., a dimension associated with sweeping bias voltages and/or sweeping time-varying voltages at each bias voltage step, among other examples), a time series dimension 610, a feature dimension 615 (e.g., one or more measurements of currents and/or voltages performed at the various bias voltages at various times, such as V_{CE} measurements, I_{WE} measurements, V_{bias} measurements, and/or similar features and/or parameters, and/or derived features and/or parameters), a batch dimension 620, and/or similar dimensions. For example, as described above in connection with FIG. 4, in some aspects a gas detection system may be associated with sweeping through multiple bias voltages (e.g., multiple steady and/or DC voltages) and, at each voltage, sweeping through multiple time-varying voltages (e.g., multiple AC voltages). Accordingly, in such aspects, the tensor schematically indicated by reference number 600 may include real-time data associated with the multiple bias voltages and/or time-varying voltages, measurements performed at each bias voltage and/or time-varying voltage, and/or similar data.

As indicated by reference number 625, data collected by the gas detection system (e.g., the tensor described above in connection with reference number 600), may be input into an ML model, which may be an ANN model and/or a deep-learning model, among other examples. For example, the ML model may be associated with multiple ML steps, layers, and/or sub-models used to generate an output, such as probabilities that one or more gases are present at the electrochemical devices and/or estimated concentrations of one or more gases at the electrochemical device. For example, in the aspect shown in connection with reference number 625, the ML model may be associated with a first series of layers (such as a first quantity of passes (shown as “n1,” where n1≥1) through a series of layers including a strided convolution two-dimensional (Conv2D) layer, a rectified linear unit (ReLU) layer, and/or a batch normalization (BN) layer, among other examples), as indicated by reference number 630, and/or a second series of layers (such as a second quantity of passes (shown as “n2,” where n2≥1) through a fully-connected (FC) layer, a third quantity of passes (shown as “n3,” where n3≥1) through a ReLU layer, and/or a dropout layer, among other examples), as indicated by reference number 635. In some other aspects, an ML model used to process data collected by the gas detection system may be associated with more or fewer layers and/or series of layers, more or fewer passes through each layer and/or series of layers, and/or different types of layers and/or series of layers without departing from the scope of the disclosure.

In such examples, the ML model may be configured to return a probability that one or more gases are present at the electrochemical device and/or an estimated concentration of each gas present at the electrochemical device. For example, as indicated by reference number 640, in aspects in which the gas detection system and/or electrochemical device are used to detect four target gases, the output of the ML model may be an array indicating a probability that each gas in present at the electrochemical device, such as a probability that CO is present (shown in FIG. 6 as p_{CO}), a probability that HCHO is present (shown in FIG. 6 as p_{HCHO}), a probability that SO₂ is present (shown in FIG. 6 as p_{SO}_{₂}), and/or a probability that NO₂ is present (shown in FIG. 6 as p_{NO}_{₂}).

In some other aspects, the ML model may be configured with a probability threshold, and thus an output of the ML model may indicate whether a gas is present based on whether the calculated probability for the gas satisfies the threshold. For example, the ML model may output a “0” for any gas for which a corresponding probability does not satisfy a probability threshold (indicative that the gas is likely not present at the sensor), and/or may output a “1” for any gas for which a corresponding probability satisfies a probability threshold (indicative that the gas likely is present at the sensor).

Based at least in part on using a multi-bias scheme in a gas detection system (e.g., gas detection system 200), improved gas detection capabilities and/or concentration calculations may be realized as compared to using a single-bias scheme, among other examples. For example, in some aspects, using a single-bias scheme with a steady voltage of one of −2V₀ mV, −V₀ mV, 0 mV, V₀ mV, or 2V₀ mV may result in a classification accuracy of less than 90%, while using a multi-bias scheme in which a steady voltage is swept through −2V₀ mV, −V₀ mV, 0 mV, V₀ mV, or 2V₀ mV, as described above in connection with FIGS. 4-6, may result in a classification accuracy of approximately 99.8%.

As indicated above, FIG. 6 is provided as an example. Other examples may differ from what is described with respect to FIG. 6.

FIG. 7 is a diagram illustrating an example process 700 performed, for example, at an electrochemical device or an apparatus of an electrochemical device, and/or a gas detection system or an apparatus of a gas detection system, in accordance with the present disclosure. Example process 700 is an example where the apparatus, the electrochemical device (e.g., electrochemical device 100 and/or the electrochemical cell 210), and/or the gas detection system (e.g., gas detection system 200) performs operations associated with a multi-bias scheme for an electrochemical gas sensor.

As shown in FIG. 7, in some aspects, process 700 may include receiving, via one or more gas apertures in an electrochemical device, a composition of one or more gases (block 710). For example, the gas detection system (e.g., using electrochemical cell 210 and/or electrochemical device 100) may receive, via one or more gas apertures in the electrochemical device, a composition of one or more gases, as described above.

As further shown in FIG. 7, in some aspects, process 700 may include applying, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage (block 720). For example, the gas detection system (e.g., using controller 205, processing system 250, waveform generator component 230, and/or the feedback loop 220) may apply, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage, as described above.

As further shown in FIG. 7, in some aspects, process 700 may include detecting at least one of a current or a voltage while applying the set of bias voltage sub-cycles, resulting in at least one of a detected current or a detected voltage (block 730). For example, the gas detection system (e.g., using controller 205, processing system 250, and/or the TIA 215) may detect at least one of a current or a voltage while applying the set of bias voltage sub-cycles, resulting in at least one of a detected current or a detected voltage, as described above.

As further shown in FIG. 7, in some aspects, process 700 may include identifying at least one of a presence of each gas, of the one or more gases, or a concentration of each gas, of the one or more gases, based at least in part on the at least one of the detected current or the detected voltage (block 740). For example, the electrochemical device (e.g., using controller 205, processing system 250, AI/ML component 235, and/or linear algebra component 240) may identify at least one of a presence of each gas, of the one or more gases, or a concentration of each gas, of the one or more gases, based at least in part on the at least one of the detected current or the detected voltage, as described above.

Process 700 may include additional aspects, such as any single aspect or any combination of aspects described below and/or in connection with one or more other processes described elsewhere herein.

In a first aspect, the at least one of the detected current or the detected voltage includes at least one of a current detected between the one or more working electrodes and one or more counter electrodes, a voltage detected between the one or more working electrodes and the one or more reference electrodes, a voltage detected between the one or more counter electrodes and the one or more reference electrodes, or a voltage detected between the one or more counter electrodes and the one or more working electrodes.

In a second aspect, alone or in combination with the first aspect, the plurality of time-varying voltages are associated with a plurality of alternating-current voltages, and, for each bias voltage sub-cycle, of the set of bias voltage sub-cycles, a frequency of each alternating-current voltage, of the plurality of alternating-current voltages, differs from frequencies of all other alternating-current voltages, of the plurality of alternating-current voltages.

In a third aspect, alone or in combination with one or more of the first and second aspects, each time-varying voltage, of the plurality of time-varying voltages, is associated with a plurality of voltage levels, and, for each bias voltage sub-cycle, of the set of bias voltage sub-cycles, a duration associated with each voltage level, of the plurality of voltage levels, of each time-varying voltage, of the plurality of time-varying voltages, differs from durations associated with each voltage level, of the plurality of voltage levels, of all other time-varying voltages, of the plurality of time-varying voltages.

In a fourth aspect, alone or in combination with one or more of the first through third aspects, process 700 includes square-wave voltage waveforms, sine-wave voltage waveforms, sawtooth-wave voltage waveforms, triangular-wave voltage waveforms, step-wave voltage waveforms, pulse-wave voltage waveforms, ramp-wave voltage waveforms, exponential-wave voltage waveforms, or modulating-wave voltage waveforms.

In a fifth aspect, alone or in combination with one or more of the first through fourth aspects, identifying the at least one of the presence of each gas or the concentration of each gas includes using a machine learning model to calculate probabilities that the one or more gases are present in the composition of one or more gases.

In a sixth aspect, alone or in combination with one or more of the first through fifth aspects, calculating probabilities that one or more gases are present in the composition of one or more gases is based at least in part on the at least one of the detected current or the detected voltage while time-varying voltages are applied between the one or more reference electrodes and the one or more working electrodes.

In a seventh aspect, alone or in combination with one or more of the first through sixth aspects, identifying the at least one of the presence of each gas or the concentration of each gas includes using a machine learning model to calculate the concentration of each gas.

In an eighth aspect, alone or in combination with one or more of the first through seventh aspects, identifying the at least one of the presence of each gas or the concentration of each gas includes using a linear-algebra-based algorithm to estimate the concentration of each gas.

In a ninth aspect, alone or in combination with one or more of the first through eighth aspects, using the linear-algebra-based algorithm to estimate the concentration of each gas is based at least in part on at least one of the at least one of the detected current or the detected voltage while steady voltages are applied between the one or more reference electrodes and the one or more working electrodes, or the at least one of the detected current or the detected voltage while time-varying voltages are applied between the one or more reference electrodes and the one or more working electrodes.

In a tenth aspect, alone or in combination with one or more of the first through ninth aspects, applying the set of bias voltage sub-cycles includes at least of using a potentiostat to control a voltage between the one or more reference electrodes and the one or more working electrodes, or using a galvanostat to control a current between the one or more working electrodes and one or more counter electrodes.

Although FIG. 7 shows example blocks of process 700, in some aspects, process 700 may include additional blocks, fewer blocks, different blocks, or differently arranged blocks than those depicted in FIG. 7. Additionally, or alternatively, two or more of the blocks of process 700 may be performed in parallel.

The following provides an overview of some Aspects of the present disclosure:

Aspect 1: A method of gas detection performed by an electrochemical device, comprising: receiving, via one or more gas apertures in the electrochemical device, a composition of one or more gases; applying, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage; detecting at least one of a current or a voltage while applying the set of bias voltage sub-cycles, resulting in at least one of a detected current or a detected voltage; and identifying at least one of a presence of each gas, of the one or more gases, or a concentration of each gas, of the one or more gases, based at least in part on the at least one of the detected current or the detected voltage.

Aspect 2: The method of Aspect 1, wherein the at least one of the detected current or the detected voltage includes at least one of: a current detected between the one or more working electrodes and one or more counter electrodes, a voltage detected between the one or more working electrodes and the one or more reference electrodes, a voltage detected between the one or more counter electrodes and the one or more reference electrodes, or a voltage detected between the one or more counter electrodes and the one or more working electrodes.

Aspect 3: The method of any of Aspects 1-2, wherein the plurality of time-varying voltages are associated with a plurality of alternating-current voltages, and wherein, for each bias voltage sub-cycle, of the set of bias voltage sub-cycles, a frequency of each alternating-current voltage, of the plurality of alternating-current voltages, differs from frequencies of all other alternating-current voltages, of the plurality of alternating-current voltages.

Aspect 4: The method of any of Aspects 1-3, wherein each time-varying voltage, of the plurality of time-varying voltages, is associated with a plurality of voltage levels, and wherein, for each bias voltage sub-cycle, of the set of bias voltage sub-cycles, a duration associated with each voltage level, of the plurality of voltage levels, of each time-varying voltage, of the plurality of time-varying voltages, differs from durations associated with each voltage level, of the plurality of voltage levels, of all other time-varying voltages, of the plurality of time-varying voltages.

Aspect 5: The method of any of Aspects 1-4, wherein, for each bias voltage sub-cycle, of the set of bias voltage sub-cycles, the plurality of time-varying voltages are associated with at least one of: square-wave voltage waveforms, sine-wave voltage waveforms, sawtooth-wave voltage waveforms, triangular-wave voltage waveforms, step-wave voltage waveforms, pulse-wave voltage waveforms, ramp-wave voltage waveforms, exponential-wave voltage waveforms, or modulated-wave voltage waveforms.

Aspect 6: The method of any of Aspects 1-5, wherein identifying the at least one of the presence of each gas or the concentration of each gas includes using a machine learning model to calculate probabilities that the one or more gases are present in the composition of one or more gases.

Aspect 7: The method of Aspect 6, wherein calculating probabilities that one or more gases are present in the composition of one or more gases is based at least in part on the at least one of the detected current or the detected voltage while time-varying voltages are applied between the one or more reference electrodes and the one or more working electrodes.

Aspect 8: The method of any of Aspects 1-7, wherein identifying the at least one of the presence of each gas or the concentration of each gas includes using a machine learning model to calculate the concentration of each gas.

Aspect 9: The method of any of Aspects 1-8, wherein identifying the at least one of the presence of each gas or the concentration of each gas includes using a linear-algebra-based algorithm to estimate the concentration of each gas.

Aspect 10: The method of Aspect 9, wherein using the linear-algebra-based algorithm to estimate the concentration of each gas is based at least in part on at least one of: the at least one of the detected current or the detected voltage while steady voltages are applied between the one or more reference electrodes and the one or more working electrodes, or the at least one of the detected current or the detected voltage while time-varying voltages are applied between the one or more reference electrodes and the one or more working electrodes.

Aspect 11: The method of any of Aspects 1-10, wherein applying the set of bias voltage sub-cycles includes at least of: using a potentiostat to control a voltage between the one or more reference electrodes and the one or more working electrodes, or using a galvanostat to control a current between the one or more working electrodes and one or more counter electrodes.

Aspect 12: An apparatus for wireless communication at a device, the apparatus comprising one or more processors; one or more memories coupled with the one or more processors; and instructions stored in the one or more memories and executable by the one or more processors to cause the apparatus to perform the method of one or more of Aspects 1-11.

Aspect 13: An apparatus for wireless communication at a device, the apparatus comprising one or more memories and one or more processors coupled to the one or more memories, the one or more processors configured to cause the device to perform the method of one or more of Aspects 1-11.

Aspect 14: An apparatus for wireless communication, the apparatus comprising at least one means for performing the method of one or more of Aspects 1-11.

Aspect 15: A non-transitory computer-readable medium storing code for wireless communication, the code comprising instructions executable by one or more processors to perform the method of one or more of Aspects 1-11.

Aspect 16: A non-transitory computer-readable medium storing a set of instructions for wireless communication, the set of instructions comprising one or more instructions that, when executed by one or more processors of a device, cause the device to perform the method of one or more of Aspects 1-11.

Aspect 17: A device for wireless communication, the device comprising a processing system that includes one or more processors and one or more memories coupled with the one or more processors, the processing system configured to cause the device to perform the method of one or more of Aspects 1-11.

Aspect 18: An apparatus for wireless communication at a device, the apparatus comprising one or more memories and one or more processors coupled to the one or more memories, the one or more processors individually or collectively configured to cause the device to perform the method of one or more of Aspects 1-11.

The foregoing disclosure provides illustration and description but is not intended to be exhaustive or to limit the aspects to the precise forms disclosed. Modifications and variations may be made in light of the above disclosure or may be acquired from practice of the aspects. No element, act, or instruction described herein should be construed as critical or essential unless explicitly described as such.

It will be apparent that systems or methods described herein may be implemented in different forms of hardware or a combination of hardware and software. The actual specialized control hardware or software used to implement these systems or methods is not limiting of the aspects. Thus, the operation and behavior of the systems or methods are described herein without reference to specific software code, because those skilled in the art will understand that software and hardware can be designed to implement the systems or methods based, at least in part, on the description herein. A component being configured to perform a function means that the component has a capability to perform the function, and does not require the function to be actually performed by the component, unless noted otherwise.

As used herein, the articles “a” and “an” are intended to refer to one or more items and may be used interchangeably with “one or more” or “at least one.” Further, as used herein, the article “the” is intended to include one or more items referenced in connection with the article “the” and may be used interchangeably with “the one or more.” Furthermore, as used herein, the terms “set” and “group” are intended to include one or more items and may be used interchangeably with “one or more.” Where only one item is intended, the phrase “only one” or “a single one” or similar language is used. Also, as used herein, the terms “has,” “have,” “having,” “comprise,” “comprising,” “include” and “including,” and derivatives thereof or similar terms are intended to be open-ended terms that do not limit an element that they modify (for example, an element “having” A may also have B). Also, as used herein, the term “or” is intended to be inclusive when used in a series and may be used interchangeably with “and/or,” unless explicitly stated otherwise (for example, if used in combination with “either” or “only one of”). As used herein, a phrase referring to “at least one of” a list of items refers to any combination of those items, including single members. As an example, “at least one of: a, b, or c” is intended to cover a, b, c, a+b, a+c, b+c, and a+b+c, as well as any combination with multiples of the same element (for example, a+a, a+a+a, a+a+b, a+a+c, a+b+b, a+c+c, b+b, b+b+b, b+b+c, c+c, and c+c+c, or any other ordering of a, b, and c).

As used herein, the term “determine” or “determining” encompasses a wide variety of actions and, therefore, “determining” can include calculating, computing, processing, deriving, estimating, investigating, looking up (such as via looking up in a table, a database, or another data structure), searching, inferring, ascertaining, and/or measuring, among other possibilities. Also, “determining” can include receiving (such as receiving information), accessing (such as accessing data stored in memory) or transmitting (such as transmitting information), among other possibilities. Additionally, “determining” can include resolving, selecting, obtaining, choosing, establishing, and/or other such similar actions.

As used herein, the phrase “based on” is intended to mean “based at least in part on” or “based on or otherwise in association with” unless explicitly stated otherwise. As used herein, “satisfying a threshold” may, depending on the context, refer to a value being greater than the threshold, greater than or equal to the threshold, less than the threshold, less than or equal to the threshold, equal to the threshold, or not equal to the threshold, among other examples.

Even though particular combinations of features are recited in the claims or disclosed in the specification, these combinations are not intended to limit the scope of all aspects described herein. Many of these features may be combined in ways not specifically recited in the claims or disclosed in the specification. The disclosure of various aspects includes each dependent claim in combination with every other claim in the claim set.

What is claimed is:

1. A gas detection system, comprising:
an electrochemical device;
one or more components, coupled to the electrochemical device, configured to cause the gas detection system to:
receive, via one or more gas apertures in the electrochemical device, a composition of one or more gases;
apply, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage;
detect at least one of a current or a voltage while applying the set of bias voltage sub-cycles, resulting in at least one of a detected current or a detected voltage; and
identify at least one of a presence of each gas, of the one or more gases, or a concentration of each gas, of the one or more gases, based at least in part on the at least one of the detected current or the detected voltage.

2. The gas detection system of claim 1, wherein the at least one of the detected current or the detected voltage includes at least one of:
a current detected between the one or more working electrodes and one or more counter electrodes,
a voltage detected between the one or more working electrodes and the one or more reference electrodes,
a voltage detected between the one or more counter electrodes and the one or more reference electrodes, or
a voltage detected between the one or more counter electrodes and the one or more working electrodes.

3. The gas detection system of claim 1, wherein the plurality of time-varying voltages are associated with a plurality of alternating-current voltages, and
wherein, for each bias voltage sub-cycle, of the set of bias voltage sub-cycles, a frequency of each alternating-current voltage, of the plurality of alternating-current voltages, differs from frequencies of all other alternating-current voltages, of the plurality of alternating-current voltages.

4. The gas detection system of claim 1, wherein each time-varying voltage, of the plurality of time-varying voltages, is associated with a plurality of voltage levels, and
wherein, for each bias voltage sub-cycle, of the set of bias voltage sub-cycles, a duration associated with each voltage level, of the plurality of voltage levels, of each time-varying voltage, of the plurality of time-varying voltages, differs from durations associated with each voltage level, of the plurality of voltage levels, of all other time-varying voltages, of the plurality of time-varying voltages.

5. The gas detection system of claim 1, wherein, for each bias voltage sub-cycle, of the set of bias voltage sub-cycles, the plurality of time-varying voltages are associated with at least one of:
square-wave voltage waveforms,
sine-wave voltage waveforms,
sawtooth-wave voltage waveforms,
triangular-wave voltage waveforms,
step-wave voltage waveforms,
pulse-wave voltage waveforms,
ramp-wave voltage waveforms,
exponential-wave voltage waveforms, or
modulated-wave voltage waveforms.

6. The gas detection system of claim 1, wherein the one or more components, to cause the gas detection system to identify the at least one of the presence of each gas or the concentration of each gas, are configured to cause the gas detection system to use a machine learning model to calculate probabilities that the one or more gases are present in the composition of one or more gases.

7. The gas detection system of claim 6, wherein the one or more components, to cause the gas detection system to calculate the probabilities that one or more gases are present in the composition of one or more gases, are configured to cause the gas detection system to calculate the probabilities that one or more gases are present in the composition of one or more gases based at least in part on the at least one of the detected current or the detected voltage while time-varying voltages are applied between the one or more reference electrodes and the one or more working electrodes.

8. The gas detection system of claim 1, wherein the one or more components, to cause the gas detection system to identify the at least one of the presence of each gas or the concentration of each gas, are configured to cause the gas detection system to use a machine learning model to calculate the concentration of each gas.

9. The gas detection system of claim 1, wherein the one or more components, to cause the gas detection system to identify the at least one of the presence of each gas or the concentration of each gas, are configured to cause the gas detection system to use a linear-algebra-based algorithm to estimate the concentration of each gas.

10. The gas detection system of claim 9, wherein the one or more components, to cause the gas detection system to use the linear-algebra-based algorithm to estimate the concentration of each gas, are configured to cause the gas detection system to use the linear-algebra-based algorithm to estimate the concentration of each gas based at least in part on at least one of:
the at least one of the detected current or the detected voltage while steady voltages are applied between the one or more reference electrodes and the one or more working electrodes, or
the at least one of the detected current or the detected voltage while time-varying voltages are applied between the one or more reference electrodes and the one or more working electrodes.

11. The gas detection system of claim 1, wherein the one or more components, to cause the gas detection system to apply the set of bias voltage sub-cycles, are configured to cause the gas detection system to at least of:
use a potentiostat to control a voltage between the one or more reference electrodes and the one or more working electrodes, or
use a galvanostat to control a current between the one or more working electrodes and one or more counter electrodes.

12. A method of gas detection performed by an electrochemical device, comprising:
receiving, via one or more gas apertures in the electrochemical device, a composition of one or more gases;
applying, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage;
detecting at least one of a current or a voltage while applying the set of bias voltage sub-cycles, resulting in at least one of a detected current or a detected voltage; and
identifying at least one of a presence of each gas, of the one or more gases, or a concentration of each gas, of the one or more gases, based at least in part on the at least one of the detected current or the detected voltage.

13. The method of claim 12, wherein the at least one of the detected current or the detected voltage includes at least one of:
a current detected between the one or more working electrodes and one or more counter electrodes,
a voltage detected between the one or more working electrodes and the one or more reference electrodes,
a voltage detected between the one or more counter electrodes and the one or more reference electrodes, or
a voltage detected between the one or more counter electrodes and the one or more working electrodes.

14. The method of claim 12, wherein the plurality of time-varying voltages are associated with a plurality of alternating-current voltages, and
wherein, for each bias voltage sub-cycle, of the set of bias voltage sub-cycles, a frequency of each alternating-current voltage, of the plurality of alternating-current voltages, differs from frequencies of all other alternating-current voltages, of the plurality of alternating-current voltages.

15. The method of claim 12, wherein each time-varying voltage, of the plurality of time-varying voltages, is associated with a plurality of voltage levels, and
wherein, for each bias voltage sub-cycle, of the set of bias voltage sub-cycles, a duration associated with each voltage level, of the plurality of voltage levels, of each time-varying voltage, of the plurality of time-varying voltages, differs from durations associated with each voltage level, of the plurality of voltage levels, of all other time-varying voltages, of the plurality of time-varying voltages.

16. The method of claim 12, wherein identifying the at least one of the presence of each gas or the concentration of each gas includes using a machine learning model to calculate probabilities that the one or more gases are present in the composition of one or more gases.

17. The method of claim 16, wherein calculating probabilities that one or more gases are present in the composition of one or more gases is based at least in part on the at least one of the detected current or the detected voltage while time-varying voltages are applied between the one or more reference electrodes and the one or more working electrodes.

18. The method of claim 12, wherein identifying the at least one of the presence of each gas or the concentration of each gas includes using a linear-algebra-based algorithm to estimate the concentration of each gas.

19. The method of claim 18, wherein using the linear-algebra-based algorithm to estimate the concentration of each gas is based at least in part on at least one of:
the at least one of the detected current or the detected voltage while steady voltages are applied between the one or more reference electrodes and the one or more working electrodes, or
the at least one of the detected current or the detected voltage while time-varying voltages are applied between the one or more reference electrodes and the one or more working electrodes.

20. A non-transitory computer-readable medium storing a set of instructions for gas detection, the set of instructions comprising:
one or more instructions that, when executed by one or more processors of a gas detection system, cause the gas detection system to:
receive, via one or more gas apertures in an electrochemical device, a composition of one or more gases;
apply, between one or more reference electrodes and one or more working electrodes, a set of bias voltage sub-cycles, wherein, for each bias voltage sub-cycle, of the set of bias voltage sub-cycles, a voltage applied between the one or more reference electrodes and the one or more working electrodes alternates between a corresponding steady voltage and a plurality of time-varying voltages centered about the corresponding steady voltage;
detect at least one of a current or a voltage while applying the set of bias voltage sub-cycles, resulting in at least one of a detected current or a detected voltage; and
identify at least one of a presence of each gas, of the one or more gases, or a concentration of each gas, of the one or more gases, based at least in part on the at least one of the detected current or the detected voltage.
